(19) United States
(12) Patent Application Publication
(10) Pub. No.: US 2008/0213390 A1
(43) Pub. Date: Sep. 4, 2008

(54) Deuterium Depleted Water (Ddw) Using As Adjuvant In Cancer Therapy For Cytostatics Toxicity Reducing

(76) Inventors: Nicolae Manolescu, Bucharest (RO); Serban Constantin Valeca, Pitesti (RO); Rodica Anghel, Bucharest (RO); Ion Balanescu, Bucharest (RO); Rodin Traicu, Drobeta Turnu Severin (RO); Dumitru Marculescu, Mehedinti (RO); Ioan Stefanescu, Ramnicu Valcea (RO); Marieta Panait, Bucharest (RO); Emilia Balint, Ilfov (RO); Ioan Encut, Bucharest (RO); Manuella Militaru, Bucharest (RO); Aneta Pop, Bucharest (RO); Sabin Cinca, Bucharest (RO); Virgiliu Comisel, Bucharest (RO); Viorel Fugaru, Bucharest (RO); Corneliu Mateescu, Bucharest (RO); Iuliana Gruia, Bucharest (RO); Victoria Moraru, Bucharest (RO); Monica Nistoroiu, Bucharest (RO); Daniela Begu, Bucharest (RO); Iolanda Dumitrescu, Bucharest (RO); Maria Ghita, Bucharest (RO)

Correspondence Address: Hershkovitz & Associates, LLC, 2845 Duke Street, Alexandria, VA, 22314, US

(21) Appl. No.: 11/660,392

(22) PCT Filed: Jul. 26, 2005
(86) PCT No.: PCT/RO05/00008

§ 371 Date: Feb. 16, 2007

(51) Int. Cl. A61K 33/00; A61P 43/00

(52) U.S. Cl. 424/600

(57) ABSTRACT

A method is provided for utilizing Deuterium Depleted Water (DDW) as an adjuvant in cancer therapy and for reducing cytostatic toxicity. The method includes administration of DDW having a concentration of about 60 ppm, as a daily diet, to animals. The cancer therapy can include a mono-chemotherapy where a single drug or cytostatic is used or a poly-chemotherapy where more than one drug or cytostatic is used. The drug or cytostatic that are used for chemotherapy can include one or more of Cyclophosphamide, 5-Flourouracil, Farmarubicine and Vinblastine. The method can be used to treat different types of cancer.

### BACKGROUND OF THE INVENTION

1. Field of the Invention

The present disclosure relates to utilizing Deuterium Depleted Water (DDW) as a reducer of cytostatic toxicity.

2. Discussion of Background Information

It is well known that when treating cancer, chemotherapy can be used both on humans and animals. Besides the effects obtained when treating tumours, chemotherapy has toxic immune-suppression effects on patients, or under certain conditions, it can even trigger secondary cancers. This is why researchers focused on finding medical methods or other natural methods to reduce cytostatic toxicity, and consequently to improve therapy and to indirectly improve the cancer patients' life condition.

The positive results of using procaine as an adjuvant to colorectal cancer chemotherapy is well known (Europ. Journal of Cancer, 1995, 31A, p. 1283-1287). Sergio Caffagi, Mauro Exposito et al. have synthesized a new cytostatic—CIS-diamino-platinum—(II) that contains procaine chloral hydrate, which is a kind of cytostatic with anti-tumor effect both in vivo and in vitro (Anticancer research, 1992, 12, p. 2285-2292). In vitro and in vivo, this new cytostatic has a renal toxicity much lower than classic cis-platinum cytostatics, and this is the reason why it is used in hospitals of oncology.

### SUMMARY OF THE INVENTION

A method to establish in vivo the efficient Deuterium Depleted Water concentration for cancer therapy on lab animals is provided. It is ascertained, by this method, that continuously administering Deuterium Depleted Water (DDW), with a concentration of 60 ppm, over a period of 60 days prior to tumor grafting, and then administering this water over an extremely long period (700 days), inhibits some experimental malign tumor development and growth of Wistar outbred rats, and finally resulting in cancer significant percentage ingrowths, as well as in significant prolongation of survival time of the animals having tumors (due to a very slow tumor growth).

The problem solved by this disclosure is finding a therapeutic composition or a method that prevents or reduces the toxic side effects of cancer chemotherapy.

Therefore, this disclosure provides a new method of utilizing or applying DDW comprising administering DDW having a concentration of 60 ppm Deuterium, as a daily diet, that allows a visible decrease of some cytostatic toxic effects on the hepatic, renal and hematopoietic areas. According to various aspects of the disclosure, the method comprises utilizing Deuterium Depleted Water having a concentration of 60 ppm Deuterium as an adjuvant of chemotherapy.

Some advantages of administering Deuterium Depleted Water during mono-chemotherapy, according to this disclosure are the following:

It reduces the toxic effects within cyto-hematological status, both on peripheral blood and on lympho-nodal areas;

It has a hepato-protective effect;

It reduces the intensity of degenerative nephritis modifications generated by cytostatics on the kidneys level;

It reduces P450 enzyme activity involved in cytostatic metabolization;

It directly minimizes glycolysis and indirectly minimizes serum glycoprotein concentration, which demonstrates that the cytostatic toxicity

It lowers the level of glutation-S-transferazes (GST) within the cytostatic metabolization, wherein a lower level of these enzymes is obtained after administering DDW which shows the positive role this kind of water has in influencing the toxicity minimization of cytostatic products; and

It has a protective effect against oxidizing processes, with a reduction of oxidizing stress factor.

Accordingly, one aspect of this disclosure is providing a method of utilizing deuterium depleted water for the manufacture of a medicament for reducing the toxicity of cytostatics by administering of water having 60 ppm concentration, before, during and after mono-chemotherapy with cyclophosphamide, or 5-fluorauracil, or farmarubicine or vinblastine, as a daily diet.

Another aspect of this disclosure is providing a method of utilizing deuterium depleted water for the manufacture of a medicament for reducing the toxicity of cytostatics by administering of water having 60 ppm concentration, before, during and after poly-chemotherapy with cyclophosphamide, or 5-fluorauracil, or vincristine or vinblastine, as a daily diet.

Still another aspect of this disclosure is providing a method of treating an organism having cancer comprising administering deuterium depleted water, before, during and after chemotherapy, as a daily diet. The organism can be an animal, such as a pet, a dog, a cat, or a human. The chemotherapy can be mono-chemotherapy or poly-chemotherapy. The chemotherapy can comprise administering one or more of, cyclophosphamide, or 5-fluorauracil, or farmarubicine and vinblastine. The chemotherapy can target the hepatic, renal or hematopoietic areas. The deuterium depleted water can have a concentration of 60 ppm.

### BRIEF DESCRIPTION OF THE DRAWINGS

Herein below is an example of utilizing DDW as an adjuvant to minimize cytostatic toxicity wherein the figures show:

FIG. 1: ³H-thymidine distribution in thighbone marrow of adult Wistar rats, after 5 days from the last cytostatic administering;

FIG. 2: ³H-thymidine distribution in thighbone marrow of adult Wistar rats, after 10 days from the last cytostatic administering;

FIG. 3: ³H-thymidine distribution in lymphnodes of adult Wistar rats, after 5 days from the last cytostatic administering; and

FIG. 4: ³H-thymidine distribution in lymphnodes of adult Wistar rats, after 10 days from the last cytostatic administering.

### DETAILED DESCRIPTION OF THE PRESENT INVENTION

60 ppm concentration Deuterium Depleted Water was used for healthy adult outbred Wistar rats. Mono-chemotherapy was used to treat these animals using the following cytostatics: Vinblastine (VBL); Cyclophosphamide (CFS); 5-Fluorouracil (5-Fu); Farmarubicine (FARM). The administered doses were:

VBL—0.1 mg/kg body weight;

CFS—5 mg/kg body weight;

5-Fu—10 mg/kg body weight; and

FARM—1 mg/kg body weight.

The cytostatics were intra-peritoneally administered (i.p.) for 5 days consecutively. The doses were established depending on the lethal dose of 50% (LD50).

The animals were distributed into two groups, as:

1) animals that had received tap water, as a daily diet, before the beginning of the cytostatic therapy, during cytostatic therapy and after the last cytostatic dose (TW (tap water)—control group); and

2) animals that had received 60 ppm Deuterium Depleted Water, as a daily diet, before the beginning of the cytostatic therapy, during cytostatic therapy and after the last cytostatic dose (DDW (Deuterium Depleted Water)—control group).

Animal sacrifice occurred over 5 and 10 days, respectively from the last cytostatic dose. After animal sacrifice, some tests were performed: bio-chemical, cyto-histological and morphological tests (on liver, spleen, kidneys), and tests for 3HTdR incorporation into lymphoid organs (bone marrow and lympho-nodules).

The four cytostatics have as a side effect of myelinic suppression, and this is why the cyto-hematologic test was performed on smears of peripheral blood from hemato-generating marrow and from lymphatic ganglions.

The heart, the liver, the kidneys and the intestines of the animals were also morphologically examined. The slides were May-Grünvald-Giemsa stained.

Biochemical exams comprised sialic acid pouring into the serum extracted from the whole blood; lipid peroxidizing (MDA); and oxidative stress factor determination (ISO).

Also, the enzymes involved in cytostatics metabolization (P450-cytocromes, triggering enzymes, GST-glutation S-transferaze) were examined, as well.

Proteins; glycoproteins; the extent of glycolysis involving serum proteins; and the electrophoresis of these serum-proteins in agarose gel were examined.

³HTdR incorporation was identified in thighbone marrow and in the lymphonodules of healthy adult Wistar rats.

All of these examinations were performed both on the DDW-control group and on the TW-control group.

A. Health Development of the Animals in the Two Groups: DDW and TW-Control

The protective effect of the 60 ppm Deuterium Depleted Water against the toxicity generated by cytostatics were studied using lots of 88 Wistar rats, each lot, males and females, with an average weight of 172 g. The animal lots were established as per GLP on pharmacological studies on laboratory-tested animals.

The possibility of acute cytostatic toxicity reduction by DDW administering as animals' daily diet was noted.

There were several important side effects. Decrease of hematopoiesis of immunological areas of the body was the most important side effect, with myelin suppression being the limiting factor for cytostatic dose, since the 4 types of cytostatics have the main side effect of the myelin suppression. The lowering of the level of this toxic phenomenon of the rats in the DDW-control group was observed, comparing to the level of this toxic phenomenon of the animals in the TW-control group.

The life quality condition of the rats was observed with respect to the improvement of this factor of the DDW group, through decrease or elimination of the other side effects that accompany administration of cytostatics when compared to the TW-control group.

Thus, it is known that the most serious adverse reactions accompanying the administration of cytostatics occur to the:
a) digestive system: the 4 types of cytostatics can create nausea, vomit, inflammations of mucous membranes (from stomatitis to ulceration, depending on the administered dose), anorexia, diarrhea, or constipation;
b) urinary system: cyclophosphamide can produce micro and macroscopic hematurias;
c) cardiovascular and pulmonary systems: cyclophosphamide can produce pneumonia and late pulmonary fibrosis; cyclophosphamide, 5-fluorouracil and farmarubicine can produce cardiac insufficiency depending on the administered dose;
d) skin: all of the cytostatics can produce alopecia, including nail and skin pigmentations; and
e) peripheral and central nervous systems: 5-fluorouracil and vinblastine can produce ataxia, paresthesia, and peripheral neuritis.

All of these manifestations have been observed in both of the two groups of animals.

Other observed parameters were:

spontaneous mortality rate;

body weight loss;

digestive and nasal hemorrhage;

apathy; and

anorexia.

The observation of the above parameters has led to the following findings:
of the DDW group animals receiving cyclophosphamide, 5-fluorouracil or farmarubicin, there were no records of any spontaneous dead animals. Within this group of animals there were no signs of toxicity during the 5 days of therapy, or during the post-therapy period. Of the TW-control group being similarly treated, spontaneous mortality occurred and it was necessary to sacrifice the animals that showed obvious toxicity signs.
of the DDW group animals receiving vinblastine (in this case, the most toxic cytostatic), low intensity toxic effects and a low percentage of mortality were recorded (25% versus 66% at TW-control group).
body weight increase demonstrated some differences between the two groups of animals. Of the TW-control group, a body weight decrease was recorded, until the 5^{th} day of the post-therapy period. Of the DDW group receiving vinblastine, the animals' body weight increase was far more evident when compared to the TW-control group;
the clinical condition of the animals demonstrated that in the case of the DDW group, no cytostatic typical toxicity phenomena occurred (except for the group receiving vinblastine, when some low intensity phenomena occurred), as compared to the TW group that demonstrated obvious toxicity phenomena.

B. Biochemical Examinations

a) Sialic Acid Dosing

Taking into consideration the importance of sialic acid (N-acetil-neuraminic acid) in manifesting the cell biological properties, the quantitative changes of this acid were studied from the DDW animals group that received, as a daily diet, Deuterium Depleted Water before the beginning of the cytostatic therapy, during this therapy and also after the last cytostatic dose, and were compared to those of the animals of the TW-control group.

The study was performed on a number of 32 Wistar outbred rats.

For each sampled serum quantity, sialic acid was dosed after serum hydrolysis with 0.1N sulphuric acid, using the Kattermann micro-method. The free or bonded sialic acid dosage was based on oxidation of sialic acid with periodic acid, and on the mono-aldehydes generation that combined with 2-sulph-barbituric 2-thiobarbituric acid which produced a stain compound that was spectrophotometrically measured.

The sialic acid level was examined and quantified from the serum of the Wistar rats. The findings are shown in Table 1.

TABLE 1

| | | | | DDW Group |
|---|---|---|---|---|
| | | | TW-control | (daily diet with |
| | | | Group | Deuterium |
| | | | Sialic acid | Depleted Water) |
| | | | mmol/l | Sialic acid |
| Lot | | Number of | (average | mmol/l |
| No. | Cytostatic | rats | values) | (average values) |
| I | Cyclophosphamide | 8 | 2.77 | 2.68 |
| II | 5-fluorouracil | 8 | 3.04 | 2.43 |
| III | farmarubicine | 8 | 2.88 | 2.62 |
| IV | vinblastine | 8 | 3.00 | 2.82 |

The findings regarding average values of sialic acid levels shown in the above Table 1 demonstrate that for all of the administered cytostatics, the sialic acid levels are lower in the DDW group.

b) Lipids Peroxiding

Amongst the biological effects produced by free radicals, the high degree of lipid peroxidation can be explained also by a higher probability for the reactive species of oxygen to encounter lipid compounds, elements of cell membranes or cell organelles. The lipid peroxidation is a typical example of a reaction having a chain radical mechanism. A single peroxil radical that can react with a fatty poly-unsaturated acid could not only result in altering the structure of the acid, but it could generate also a series of fatty acid peroxil radicals. These would, then, interact with other lipids, keeping active the transfer reaction of one electron, and the oxidation would destroy the substratum. The toxic aldehydes resulted from lipids in cell membranes peroxidation have their own biological effects. Being unsaturated α-β, they are electrophilic species with a high reactivity, and easily interact with —SH groups of the proteins or with the thiols having small molecular mass (glutathione), making the proteins and enzymes with essential —SH groups inactive. Thus, a series of cyto-pathological consequences of the lipid peroxidation in membranes are explainable.

The measurements were made, keeping in mind the oxidative stress parameter modifications induced to the two groups of animals—TW-control and DDW—due to the cytostatics administering.

Therefore, measurements of the reactions of lipidic peroxidizing, thiol-oxidizing, and murine ceruloplasmin measurement were performed. These measurements were determined to be important to show oxidative stress. This was determined by the reaction between 2thiobarbituric (TBA) and malondialdehyde (MDA) as index of lipid peroxidation and ceruloplasmin dosing.

TBA reacting materials generation is frequently used to show in which organs it is possible for lipid peroxidation to occur. The lipid peroxidation of the biological samples can be shown by measurement of Malondialdehyde (MDA) formation as a result of endoperoxides breaking down during the last stage of poly-unsaturated fatty acids oxidation. This method is the most simple one and presently, the most used. MDA represents the final product of lipid peroxidation and is the by-product of prostaglandins and thromboxanes biosynthesis.

Ceruloplasmine was dosed into extra-cell fluids by the Ravin method. This dosing method was chosen to illustrate the benefits derived from using the method according to the present invention because ceruloplasmine is able to catalyze the oxidation of Fe²⁺ to Fe³⁺ after it is released from transferrin. This activity is a mechanism by which the lipid peroxidation and free radicals generation promoted by iron ions can be prevented. It is known that lipid peroxidation can be initiated by the transit metals ions, especially by the iron and copper ions which are ubiquitous in the biological systems. Any increase of iron and copper salts concentration will result in tissue degradation and would enhance the oxygen toxic effects. Ceruloplasmine inhibits the redox cycles which are necessary for toxic effects initiation.

A new parameter was used to show the benefits derived by using the method according to this disclosure. To show oxidative stress, this new parameter was used, namely the oxidative stress factor, which is defined as being the ratio between oxidative reactions and the anti-oxidant reactions. These reactions are measurable by way of ceruloplasmine and lipid peroxides values referred to as the thiol-oxidation basis. Thus, the oxidative factor could be calculated using the formula:

$$Oxidative Stress Factor ( O S F ) = \frac{\begin{matrix}lipid peroxides \times \\ ceruloplasmine\end{matrix}}{thiol \text{-} albuminous groups} \underline{.}$$

The obtained results are shown in Table 2 below.

TABLE 2

| | | | | Oxidative |
|---|---|---|---|---|
| | Peroxides | | Thiol- | Stress |
| | nmol/100 ml | Ceruloplasmine | albuminous | Factor |
| LOT | serum | (U.I.) | nmol/l | (OSF) |
| DDW + CFS | 5.65 | 64 | 247 | 1.46 |
| DDW + 5-FU | 5.32 | 81 | 285 | 1.51 |
| DDW + FARM | 6.25 | 69 | 397 | 1.08 |
| DDW + VBL | 5.74 | 71 | 178 | 1.28 |
| TW + CFS | 6.54 | 91 | 349 | 1.70 |
| TW + 5-FU | 6.47 | 63 | 267 | 1.52 |
| TW + FARM | 6.14 | 110 | 204 | 3.31 |
| TW + VBL | 7.48 | 80 | 358 | 2.98 |

Table 2 shows that the Oxidative Stress Factor values are smaller for the group of animals treated with cytostatics and Deuterium Depleted Water as a daily diet, and this demonstrates that, within the ratio oxidants/anti-oxidants, the anti-oxidant branch is stronger, having effect on activating the protection system against oxidative stress accompanying the cytostatic metabolization.

c) The Study of Enzymes Involved in Cytostatic Metabolization

The enzymes involved in cytostatics metabolization have been classified as activation enzymes (or phase I enzymes) such as P450 cyto-chromes and detoxifying enzymes (or phase II enzymes) such as glutathione S-transferases (GST).

The role of activation enzymes (MFO) is to transform the hydrophobic substances into hydrophilic substances. The homo-oxygenating substances catalyze the insertion of a single oxygen atom from the oxygen molecule on an organic substratum, while the other oxygen atom is reduced to water. This process occurs due to the reactions involved in the substratum below:

AH+O₂+2NADPH→AOH+2NADP*+H₂O.

This enzymatic system pertains to the level of the endoplasmic reticulum membrane of the mitochondrion and the nucleus' envelop. To study it, the cell organelles were separated by repeated centrifugation with a final separation of microsomes at 105,000 g via ultra-centrifugation. At the microsomes level, the P450 cyto-chromes (a member of the iso-enzymes family) activity and concentration are measured, which are the most important indications of enzyme activation (MFO).

The P450 concentration is spectrophotometrically determined after CO and Natriumdithionit reduction of microsomal P450 (Omura and Sato method). As P450 is an isoenzyme family, it intervenes both in activation and in detoxification of xenobiotics in addition to cytostatics' activation and detoxifications. The enzymatic activity has been determined for a single substratum, namely for p-nitroanisol, which involves a de-metilation reaction.

Another family of enzymes involved in cytostatic detoxification is the glutathione S-transferases (GST). These are a group of enzymes that catalyze the conjugation of some electrophile products (of endogen or exogen origin) with glutathiones (GSH) according to the reaction:

RX+GSH→GS—R+HX.

In order to show GST activity, the Habrig method was used.

One of the important properties of this enzymatic system is the fact that, similar to P450 cyto-chromes, it shows a reduced substratum specificity, which is a property needed for xenobiotics detoxification, and that it also shows a high specificity of their metabolization.

Together with GST, the GSH (the main sulph-hydric non-protein compound from living tissues) intervenes within the detoxification processes, working as an electrophile “scavenger”, which is also a reaction possible under the condition of GST absence:

R⁺+GSH→R-GS+H⁺.

In mammalian cells, the GSH has a double functionality oscillating between the reduced form and the oxidized form. It takes part in the reduction of H₂O₂, which is a reaction catalyzed by glutathione peroxidase, being, therefore, involved in some normal radical or pathological processes control.

GSH was determined through the aloxane method.

The measurements results are shown in Table 3 below.

TABLE 3

| | P450 Activity | GSH | GST |
|---|---|---|---|
| | nmoles/mg | Snmoles/mg | nmoles/mg |
| | proteins/min | proteins | proteins/minute |
| CFS + TW | 0.7414 | 0.598 | 2.580 |
| CFS + DDW | 0.3999 | 0.368 | 1.096 |
| 5-FU + TW | 0.7341 | 0.598 | 1.47 |
| 5-FU + DDW | 0.4557 | 0.478 | 1.37 |
| FARM + TW | 0.6031 | 0.581 | 1.73 |
| FARM + DDW | 0.4739 | 0.419 | 1.313 |

From Table 3, the following conclusions can be drawn:
due to Deuterium Depleted Water utilization as a daily diet, some changes in the activity of the enzymes in phase I and phase II are noted comparing to TW-control group;
in the case of cyclophosphamide, which is a nonspecific form without alkylation activity, its activation occurs at the liver level under the action of P450. The administration of cyclophosphamide causes a decrease of approximately 100% of the phase I and II enzymes in the group TW-CFS;
Deuterium Depleted Water utilization together with the cyclophosphamide causes a visible diminuation of enzymes activity;
5-fluorouracil induces the phase I enzymes, enhancing the P450 activity in the group of animals treated with Deuterium depleted Water to approximately 200% (double) of that of the control group;
farmarubicine is metabolized into liver through reduction and hydrolysis. Administration of farmarubicine together with Tap Water highly induces the P450 activity and concentration of P450, but when Deuterium Depleted Water is administered, the P450 activity is much more reduced;
in all of the lots of animals treated with cytostatics and DDW, both GSH concentration and GST activity were much lowered as compared to those of the lots of TW-control animals treated with cytostatics and tap water.

d) Measurement of Protein, Glycoprotein and Serum Protein Glycolysis

The methods used for these measurements are well known (measuring burette and the (glucidic) glucose compound dosing in the reaction with orto-toluidine).

The findings of these studies are shown below in Tables 4 and 5.

TABLE 4

| | | | Amount of |
|---|---|---|---|
| | | Glucose (glucides) | Glycolysis |
| | Total protein | compound | mg glucides/g |
| | g/dL | mg/dL | protein |
| CFS + TW | 6.9 | 45.49 | 6.59 |
| CFS + DDW | 5.5 | 30.1 | 5.47 |
| 5-FU + TW | 6.6 | 45.49 | 6.89 |
| 5-FU + DDW | 6.2 | 26.27 | 4.2 |
| FARM + TW | 5.08 | 49.41 | 9.72 |
| FARM + DDW | 6.1 | 38.8 | 6.3 |
| VBL + TW | 5.15 | 41.56 | 8.06 |
| VBL + DDW | 4.5 | 34 | 7.5 |

TABLE 5

| | | Amount of | Percentage of |
|---|---|---|---|
| | Amount of | glycolysis | decrease |
| | Glycolysis | with Deuterium | of protein |
| | with Tap Water | Depleted Water | glycolysis in |
| | mg glucides/ | mg glucides/ | animals receiving |
| | g proteins | g proteins | DDW |
| cyclophosphamide | 6.59 | 5.47 | 17% |
| 5-fluorouracil | 6.89 | 4.2 | 39% |
| farmarubicine | 9.72 | 6.3 | 35% |
| vinblastine | 8.06 | 7.5 | 7% |

From Tables 4 and 5 the following conclusions can be drawn:
the 60 ppm concentration of Deuterium Depleted Water that was included in the daily diet of the animals caused the decrease in the amount of glycolysis which resulted in the decrease of the serum glycoproteins concentration; and
each cytostatic produced a different effect.

e) Measurement of Serum Proteins Gel Electrophoresis in Agarose

The method utilized comprises a double diffusion in agarose gel using two lectins from wheat seed embryo and Raphanus Sativus. The fact that the serum sampled from cytostatics and DDW-treated animals group did not interact with lectin confirms that DDW has a favorable effect both on the amount of glycolysis and on serum glycoproteins distribution.

f) Measurements of ³HTdR (³H Tritiated Thymidine)

The incorporation of ³HTdR was measured in thighbone marrow and in lympho-nodules of adult Wistar rats from both groups (DDW and TW-control).

The incorporation capacity was determined by intra-peritoneally injecting a quantity of 37 kBq/g body weight (1 μCi/g) of 3H-thymidine with a specific activity of 925 GBq/mmol (25 Ci/mmol), with a radiochemical purity of higher than 95%. The biological samples were sampled after 5 hours of injecting ³HTdR, and then were dissolved in Soluene-350, and then were re-suspended in a liquid scintillator (Hyonic fluor). The activity measurement was performed using a Tri-Carb, Packard Model device.

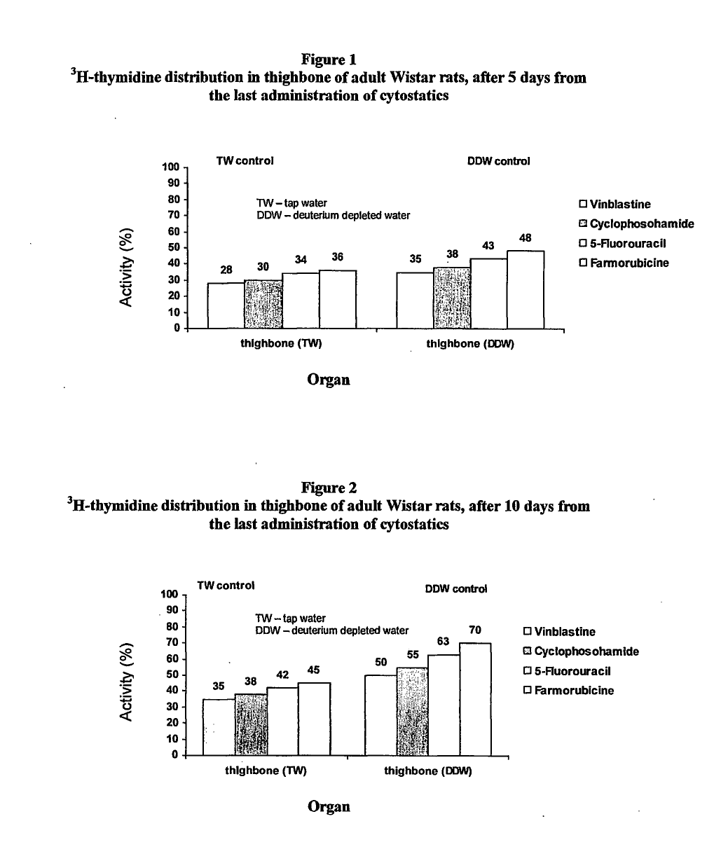

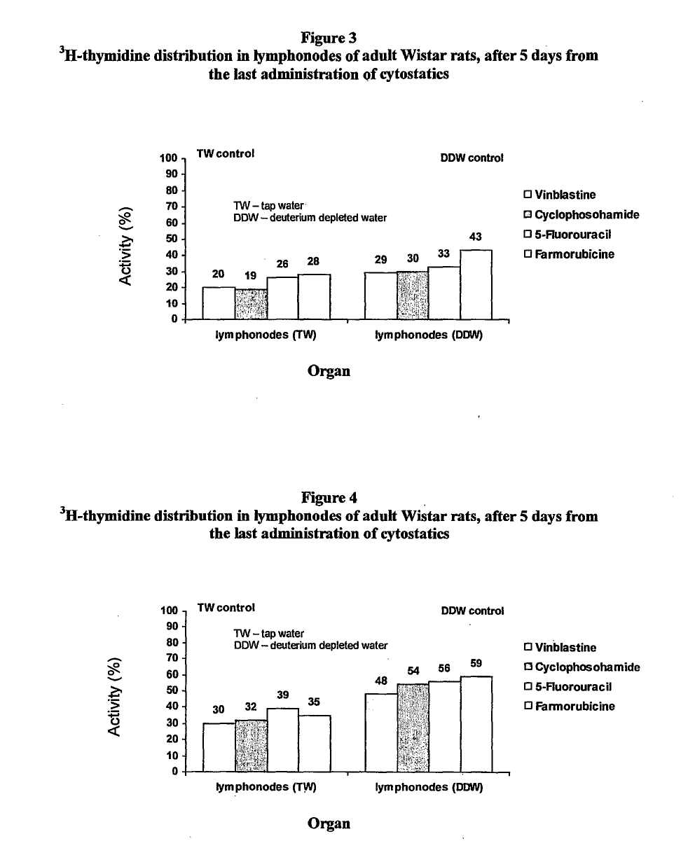

After 5 days from the last cytostatic dose, a significant percentage of increase of ³HTdR (7% and 15%) distribution was registered both in marrow and in lympho-nodules, in the animals receiving 60 ppm concentration of Deuterium Depleted Water as a daily diet, when compared to the TW-control group (FIGS. 1 and 3).

After 10 days from the last cytostatics dose, the ³HTdR distribution in bone marrow and in lympho-nodules of the DDW group of animal was more significant as compared to the TW-control group, (14%-25%) as shown in FIGS. 2 and 4.

These findings demonstrate a real improvement of AND synthesis. This significant increase of AND synthesis is also a result of reduction of cytostatic-produced toxicity through the daily administration of the 60 ppm Deuterium Depleted Water, and therefore is a beneficial application of DDW as an adjuvant for reduction of cytostatics toxicity.

C. Cyto-Hematological Studies

Studies were performed in order to show certain cyto-morphological effects within the hematopoietic area as a result of administering 60 ppm Deuterium Depleted Water as a daily diet, when compared to the TW-control group that received tap water as a daily diet.

The studies were performed using samples from peripheral blood, hematogenous marrow and lymphatic ganglions. The prepared samples were classically stained with May-Grünwald-Giemsa.

These studies led to the following findings:
the toxicity of all cytostatics used according to the studies had very high values within hematopoietic areas of the TW-control group, where 150 ppm Deuterium tap water was administered as a daily diet:
   anemia and granulopenia resulted in the peripheral blood of the cyclophosphamide-treated animals;
   leukocytosis resulted in the peripheral blood of the 5-fluorouracil-treated animals;
   leukopenia, anemia and granulopenia resulted in the peripheral blood of the farmorubicin-treated animals;
   anemia, thrombocytopenia, leukopenia and granulopenia resulted in the peripheral blood of the vinblastine-treated animals; and
   hypo-regenerative anemia resulted in the medulogram of the animals;
the administration of the 60 ppm Deuterium Depleted Water as the daily diet, significantly reduced, to almost zero, the toxic effect in the hematopoietic zona of the animals; and
the cyto-hematological reveals that the differences between the TW-control group and the DDW group is manifested both in the peripheral blood and in the lympho-nodal area, but not in the medullar zona of hematopoiesis.

D. Histological Studies

The tissue samples taken from Wistar rats were fixed in a solution of 10% formaldehyde. To show the histological changes, the Masson method using paraffin insertion and tri-chrome staining technique was used.

The microscopic aspects of histological examinations revealed the following findings:

a) Liver
in all of the animals treated with 60 ppm Deuterium Depleted Water as a daily diet (the DDW group), tissue areas with morphologically non-modified hepatocytes and extension-limited, hepatocyte degeneration zones were recorded. The degenerative modifications were manifested through a hepatocytarian tumefaction, granular dystrophy or granulo-vacuolar dystrophy and through cytoplasm oxyphiliation. All of the lesions of these types are considered to be potentially reversible;
in the DDW and vinblastine treated animals, the hepatocytary regeneration is clearly manifested; and
in the animals of the TW-control group, hepato-steatosis was found, which indicates an aggravation of the degenerative lesions.

b) Kidneys
the presence of mono-nuclear infiltration in most of the animals in the TW-control group as well as its absence under the condition of Deuterium Depleted Water administering suggests the protective effect of the DDW on the renal interstitium; and
granulo-vacuolar nephrosis was more reduced in terms of extension and intensity in the DDW group.

c) Intestine
in most of the cases from both groups of animals, no modifications were found in the intestinal walls.

d) Myocardium
no significant modifications were found in the myocardium morphology.

From histological examinations, the following conclusions can be drawn:
the administration of 60 ppm concentration Deuterium Depleted Water as a daily diet has an hepato-protective effect, with liver morphology preservation, comparing to the hepato-steatosis found in animals that were administered with tap water in their daily diet;
the hepatocytary regenerative phenomena are far more obvious when vinblastine is administered together with Deuterium Depleted Water;
the administration of 60 ppm concentration Deuterium Depleted Water as a daily diet has a protective effect on the renal interstitium; and
the administration of 60 ppm concentration Deuterium Depleted Water as a daily diet has an impact in reducing the intensity of nephrocitary degenerative modifications while nephrocitary degenerative modifications are found when normal water is administered.

The relevant results of all of the studies demonstrate that administration of 60 ppm Deuterium Depleted Water as a daily diet, to healthy adult female and male outbred Wistar rats before, during and after cytostatics administration (mono-chemotherapy) determines mainly the hepato-protective effect, which are a reduction of the toxic effect of cytostatics within the hematopoietic area, the reduction of the amount of nephrocitary degenerative modifications and the reduction of glycolysis of serum proteins. All of these results demonstrate that Deuterium Depleted Water having a 60 ppm concentration reduces the toxicity of administered cytostatics and is effective as an adjuvant in cancer chemotherapy.

Additional studies were made to demonstrate that 60 ppm DDW is a beneficial supplement in various types of cancer poly-chemotherapy on dogs.

In these studies, 26 dogs of different outbreed, sex and age, with different types of clinically and para-clinically diagnosed cancer through cyto-morphological and hematological examinations or through lympho-nodal piercing, were divided into two groups:
Group A: dogs treated with cytostatics (poly-chemotherapy as successive treatment sessions, with different periods of breaks depending on the anatomo-clinical type of cancer) were administered with 60 ppm DDW as a daily diet, with 14 days before the poly-chemotherapy, during the therapy period and after therapy completion.
Group B (control): dogs which were treated with the same type of cytostatics and in the same routine but 150 ppm Deuterium concentration of tap water (TW) were administered.

Poly-chemotherapy was applied using the following cytostatics:
1. Cyclophosphamide: 80-100 mg/m²; with a treatment session of 14 days;
2. 5-Fluorouracil or Metrotrexat: 50-75 mg/m²; with a treatment session of 7 days;
3. Vinblastin: 2-3 mg/m²; with a treatment session of 7 days; and
4. Vincristin: 1 mg/m²; with treatment session of 3 days.

All of these cytostatics are known to have toxic effects, wherein:
cyclophosphamide firstly affects the tissues having a fast turn-over, causes urinary bladder fibrosis, hyper-uremia hepato-toxic modifications, myelo-suppressive effect, leukopenia and trombocytopenia, hemorrhaging—necrotic cystitis, alopecia, etc.;
5-Fluorouracil causes medullar aplasia, nausea, vomiting, diarrhea, digestive hemorrhages, ulcers, lacrimal channel stenosis, conjunctivitis, etc.;
Vinblastin causes myelin-suppression, urinary retention, neuro-muscular and pulmonary disorders; and
Vincristin causes leucopenia, thrombocytopenia, alopecia, rash, oedema, paresthesia, loss of deep tendonal reflexes, myalgia, paraplegic ileus, polyuria or disuria, urinary retention, etc.

The 60 ppm DDW and cytostatic treated dogs manifested an almost normal appetite comparing to the dogs receiving exclusively cytostatics.

As regarding locomotion disorders (that are specific to advanced cancers), the 60 ppm DDW and cytostatic treated dogs had a muscular tonus clearly superior to the ones treated only with cytostatics. One of the explanations is that a better toxins elimination occurred through urine.

The dogs having malign lymphoma treated with 60 ppm DDW and cytostatics showed an obvious micro-poly-adenopathy compared to the macro-poly-adenophaty noted for the dogs treated only with cytostatics.

These aspects are explained also by the cytopathic effect that DDW has on malign lymphocytes.

For the group of dogs treated only with cytostatics, a series of disorders appeared, such as:
renal: glomerulonephritis; proteinuria and nephritic syndrome; Bence-Jones protein hematuria in the case of B plasmacytom;
splenomegaly clinically and echografically disorders;
mucosas might show a hemorrhagipary syndrome; micro-hemorrhages at the tongue bridle level, at the soft palate level, etc.;
tachycardia and tachypnea—signs of respiratory and systemic cardio-circulation disorders;
gastro-intestinal manifestations: constipation or diarrhea; progressive weakness (weight loss) of the animals, gastro-intestinal ulcers with melena and hematemesis etc.
skin paraneoplastic manifestations: alopecia; dermato-fibrosis; erythema; urticaria; rash; skin and mouth ulcers; and
hematological manifestations: erythrocytosis in the case of renal, liver, or pancreatic tumors; anemia in the case of pancreatic tumors and melanoma; granulocytosis in the case of melanoma and lymphoma; thrombocytosis in leukemia and carcinoma.

The severity of all of these disorders which were present in dogs having different types of cancer and undergoing poly-chemotherapy treatments, was significantly reduced when 60 ppm DDW were administered as a daily diet to the animals.

The combination of treatments comprising administration of cytostatics and 60 ppm DDW also caused a lowered level of urea and serum creatinine both during and after the therapy.

For example, for an 11 years-old, male German Shepherd dog with cellular lymphoma B diagnosis, before the cytostatic and 60 DDW therapy, the serum urea values were 58.8 mg/dL, during the therapy, they were 37.9 mg/dL and post-therapy, the values were 26.3 mg/dL. For the same animal, the serum creatinine values were 1.50 mg/dL before the therapy, 1.27 mg/dL during the therapy and 0.9 mg/dL after therapy.

For another dog, a 5 years-old, female Rottweiler breed, diagnosed with systemic centroblastic lymphoma B, that was treated with 60 ppm DDW and cytostatics, the following biochemical findings were recorded:
creatinine level: 1.48 mg/dL before therapy, 1.8 mg/dL during therapy and 1.38 mg/dL after therapy;
thrombocytes level: 135 thousands/mm³ before therapy, 209 thousands/mm³ during therapy and 201 thousands/mm³ after therapy; and
leukocytes level: 9.45 thousands/mm³ before therapy, 12.69 thousands/mm³ during therapy and 8.70 thousands/mm³ after therapy.

While for a 2.8 years-old, female Pekinese dog, diagnosed with Waldenström malady that was treated only with cytostatics, the following biochemical results were recorded:
urea level: 30.6 mg/dL before therapy, 43.24 mg/dL during therapy and 52.6 mg/dL after therapy;
alkali phosphatase level: 32 U/L before therapy and 360 U/L during therapy; and
leucocytes level: 95.07 thousands/mm³ before therapy, 43.3 thousands/mm³ during therapy and 146.05 thousands/mm³ after therapy.

Another interesting aspect is related to the 60 ppm DDW effect on immune cellular system of dogs having different cancer types and treated with cytostatics. Here, 60 ppm DDW acts both on cell clones responsible for cellular and humoral mediated immunity. The 60 ppm DDW initiates an apoptosis process that is cyto-morphologically translated by reducing the proliferative cells number, and consequently, tumor volume decreases, preventing at the same time the immediate recurrence and, thus, resulting in long-lasting remissions with positive consequences for the life condition of cancer-diagnosed animals.

Thus, the 60 ppm DDW allows cell basic compound regeneration for immune system (B lymphocyte for all of its different types, dendritic cell and NK-K cell complex).

All of these studies and examinations show post-therapeutically improvement of the immune system of these animals receiving 60 DDW and cytostatics, and therefore, their therapeutical remission is extended. All of these beneficial effects provide a better protection against cytostatic toxicity. Thus, a better therapeutic index was obtained, which results in prolongation of pets' lives and a remarkable improvement in the comfort of these animals that have different types of cancers.

The results obtained from the studies of the dogs confirm the results obtained from the studies of the rats, and show that 60 ppm DDW has certain properties for cancer organism detoxification, or for an organism that is subjected to the toxic stress generated by cytostatics (i.e., negative side effects) which are used in anti-cancer polychemotherapy.

Thus, 60 DDW can be used as an effective adjuvant, in reducing the toxicity of cytostatics that are used in cancer polychemotherapy, for all organisms including humans and pets.

1-2. (canceled)

3. A method of utilizing deuterium depleted water for the manufacture of a medicament for reducing the toxicity of cytostatics by administering of water having 60 ppm concentration, before, during and after mono-chemotherapy with at least one of cyclophosphamide, 5-fluorauracil, farmarubicine and vinblastine, as a daily diet.

4. A method of utilizing of deuterium depleted water for the manufacture of a medicament for reducing the toxicity of cytostatics by administering of water having 60 ppm concentration, before, during and after poly-chemotherapy with at least one of cyclophosphamide, 5-fluorauracil, vincristine and vinblastine, as a daily diet.

5. A method of treating an organism having cancer comprising administering deuterium depleted water, before, during and after chemotherapy, as a daily diet.

6. The method of claim 5, wherein the organism is an animal.

7. The method of claim 5, wherein the organism is a pet.

8. The method of claim 5, wherein the organism is a dog.

9. The method of claim 5, wherein the organism is a cat.

10. The method of claim 5, wherein the organism is a human.

11. The method of claim 5, wherein the organism is a Wistar rat.

12. The method of claim 5, wherein the chemotherapy is mono-chemotherapy.

13. The method of claim 5, wherein the chemotherapy is poly-chemotherapy.

14. The method of claim 5, wherein the chemotherapy comprises administering at least one of cyclophosphamide, 5-fluorauracil, farmarubicine and vinblastine.

15. The method of claim 5, wherein the chemotherapy targets at least one of the hepatic, renal and hematopoietic areas.

16. The method of claim 5, wherein the deuterium depleted water has a concentration of 60 ppm.
